(19) United States
(12) Patent Application Publication
(10) Pub. No.: US 2004/0109850 A1
(43) Pub. Date: Jun. 10, 2004

(54) Treatment of bone disorders by modulation of fgfr3

(76) Inventors: Neelam Jaiswal, Cincinnati, OH (US); Adam Houghton, Cincinnati, OH (US); Lawrence Mertz, Gaithersburg, MD (US); Darren Ji, Cincinnati, OH (US); Jonathan S. Cook, Cincinnati, OH (US); Douglas W. Axelrod, Cincinnati, OH (US)

Correspondence Address: MORGAN LEWIS & BOCKIUS LLP, 1111 PENNSYLVANIA AVENUE NW, WASHINGTON, DC, 20004, US

(21) Appl. No.: 10/450,859

(22) PCT Filed: Dec. 18, 2001
(86) PCT No.: PCT/US01/48270

(30) Foreign Application Priority Data
Dec. 18, 2000 (US) 60255882
Apr. 24, 2001 (US) 60285691
Jul. 23, 2001 (US) 60306879
Sep. 10, 2001 (US) 60317974

(51) Int. Cl. A61K 38/18

(52) U.S. Cl. 424/93.7; 514/12

(57) ABSTRACT

The present invention relates to identifying genes that are differentially regulated or expressed in bone deposition disorders. Specifically, Fibroblast Growth Factor Receptor-3 (FGFR3) has been identified as being differentially regulated during the maturation of osteoblasts and whose expression can be correlated, for example, with bone deposition disorders such as osteoporosis (including correlation with degrees of severity of the disease).

### RELATED APPLICATIONS

This application claims the benefit of U.S. Provisional Applications 60/255,882 (filed Dec. 18, 2000) and 60/285,691 (filed Apr. 24, 2001), 60/306,879 (filed Jul. 23, 2001), 60/317,974 (filed Sep. 10, 2001), all of which are herein incorporated by reference in their entirety.

### BACKGROUND OF THE INVENTION

Living bone tissue is continuously being replenished by the processes of resorption and deposition of bone matrix and minerals. This temporally and spatially coupled process, termed bone remodeling, is accomplished largely by two cell populations, osteoclasts and osteoblasts. The remodeling process is initiated when osteoclasts are recruited from the bone marrow or the circulation to the bone surface to remove a disk-shaped packet of bone producing an area of resorbed surface. A team of osteoblasts recruited to the resorbed bone surface from the bone marrow subsequently replaces the bone matrix and mineral. Among the pathological conditions associated with abnormal bone cell function is osteoporosis, a diseased characterized by reduced amounts of bone (osteopenia) and increased bone fragility. These changes can be the result of increased recruitment and activity of osteoclasts, in combination with reduced recruitment or activity of osteoblasts (Teitelbaum et al. (1997) J. Leukoc. Biol. 61, 381-388; Simonet et al. (1997) Cell 89, 309-319).

A very significant patient population that would benefit from new therapies designed to promote bone formation or inhibit resorption are those patients suffering from osteoporosis. Clinically, osteoporosis is segregated into type I and-type II. Type I osteoporosis occurs predominantly in middle aged women and is associated with estrogen loss at menopause, while osteoporosis type II is associated with advancing age. An estimated twenty to twenty-five million people are at increased risk for fracture because of site-specific bone loss. The cost of treating osteoporosis in the United States is currently estimated to be in the order of ten billion dollars per year. Demographic trends, i.e., the gradually increasing age of the United States population, suggest that these costs may increase up to three fold by the year 2020 if a safe and effective treatment is not found.

The family of proteins known as fibroblast growth factors (FGFs) and their associated receptors are of particular interest to the treatment of bone disorders such as osteoporosis. FGFs differentially bind to and activate up to four related transmembrane receptors, which in turn mediate a biological response. FGF receptors (FGFR) are members of the tyrosine kinase superfamily. To date, a high affinity binding ligand to FGFR3 has been identified as FGF9 (WO 96/41523). Previous studies have demonstrated that FGFR3 plays a significant role in various bone disorders. In addition, studies have shown that FGF9 may act as a physiological ligand in the growth plate where the growth factor inhibited terminal differentiation in rat calvaria-derived cell lines that spontaneously undergo chondrocyte differentiation in vitro (Welcsler et al. (1999) Biochem. J. 342, 677-682).

Endochondral ossification is a major mode of bone formation that occurs during fetal development as chondrocytes undergo proliferation, hypertrophy, cell death and osteoblastic replacement. Disruption of FGFR3 gene produced severe and progressive bone dysplasia with enhanced and prolonged endochondral bone growth. This growth is accompanied by expansion of proliferating and hypertrophic chondrocytes within the cartilaginous growth plate. Thus, FGFR3 appears to regulate endrochondral ossification by an essentially negative mechanism, limiting rather promoting bone growth during development (Deng et al. (1996) Cell. Tissue Res. 296, 33-43).

While the role of FGFR3 in abnormal bone formation during fetal development has been studied, investigation of its role in bone resorption and overall bone turnover has not been investigated. Bone resorption is initiated with the destruction of bone matrix by osteoclasts. Following this initial phase of bone destruction, or resorptive phase, formation of new bone protein matrix sets in. New bone proteins are deposited, and sometime later, minerals begin to be incorporated into the newly formed matrix. The formation of bone matrix and its subsequent mineralization are exclusive functions of osteoblasts.

In theory, either decreased bone formation relative to resorption or increased bone resorption relative to formation can cause the net loss of bone in osteoporosis. Control of the rate of breakdown and synthesis of new bone tissue is critical to the integrity of the skeletal structure. When the rates become unbalanced, serious conditions may result. Although there is always a net excess of bone resorption in osteoporosis, the absolute amounts of bone formation and resorption can vary from case to case.

### SUMMARY OF THE INVENTION

Few treatments are available to modulate the formation and resorption processes of bone maintenance and development. In bone disorders such as osteoporosis, it may be useful to monitor or modify the expression levels or activities of genes involved in bone formation or resorption. The present inventors have examined cell populations comprising precursor stem cells and cell populations comprising precursor stem cells that have been induced to differentiate into osteoblasts and have discovered that the expression of FGFR3 changes during this differentiation process. This change in gene expression provides a useful marker for diagnostic and prognostic uses as well as a marker that can be used for drug screening and therapeutic indications.

The invention encompasses a method of stimulating a population of stem cells to differentiate into osteoblast cells comprising contacting the population of stem cells with an effective amount of an agent which increases Fibroblast Growth Factor Receptor 3 (FGFR3) expression or activity, wherein the increase in FGFR3 protein expression or activity results in differentiation of the stem cells into osteoblast cells.

The invention also encompasses a method of increasing bone density comprising administering to an animal an effective amount of an agent which increases FGFR3 protein expression, wherein the increase in FGFR3 protein expression increases bone density in the animal. In some embodiments, the stem cell is a mesenchymal stem cell and the agent is selected from the group consisting of an FGF protein, an FGF protein fragment, an FGF-9 protein and an FGF-9 protein fragment.

The invention further encompasses a method of screening for an agent that modulates the differentiation of a population of stem cells into osteoblast cells and/or increases bone density comprising exposing to the stem cells an agent to be tested, and measuring FGFR3 expression or activity following exposure to the agent, wherein an increase in FGFR3 expression or activity is indicative of an agent capable of stimulating stem cells to differentiate into osteoblast cells and/or increasing bone density.

The invention encompasses a method of screening for an agent capable of ameliorating the effects of osteoporosis comprising exposing a population of stem cells expressing FGFR3 to the agent; and measuring FGFR3 expression or activity following exposure to the agent, wherein an increase in the level of FGFR3 expression or activity is indicative of an agent capable of ameliorating the effects of osteoporosis. In some embodiments the stem cell is a mesenchymal stem cell.

The method also encompasses a method of diagnosing a condition characterized by abnormal stem cell differentiation and/or bone density comprising detecting in a stem cell sample the level of FGFR3 expression or activity wherein abnormal FGFR3 expression or activity is indicative of a condition characterized by abnormal stem cell differentiation and/or bone density. The invention also includes a method of diagnosing a condition characterized by an abnormal rate of osteoblast formation comprising detecting in a stem cell sample the level of FGFR3 expression or activity, wherein differential FGFR3 expression or activity is indicative of an abnormal rate of formation of osteoblasts. In some embodiments, the condition is osteoporosis and the stem cell is a mesenchymal stem cell.

The invention further encompasses a method of treating a patient with a condition characterized by an abnormal rate of osteoblast formation, and/or bone density comprising administering to the patient with decreased FGFR3 expression or activity a pharmaceutical composition which increases FGFR3 expression or activity. In a related aspect, the invention includes a method of treating a patient with osteoporosis comprising administering to the patient a pharmaceutical composition wherein the pharmaceutical composition alters FGFR3 expression or activity. In some embodiments, the method further comprises the step of identifying a patient with decreased FGFR3 expression in a stem cell sample prior to administering the pharmaceutical composition by while in another embodiment it further comprises the step of comparing FGFR3 expression in the stem cell sample to FGFR3 expression in a stem cell sample from the patient taken before treatment with the pharmaceutical composition.

### BRIEF DESCRIPTION OF THE DRAWINGS

FIG. 1 is a graph displaying the experimental data demonstrating an increase in FGFR3 expression following treatment with BMP-2 over a time span of forty-eight hours (open circles=control, closed circles=BMP-2).

FIG. 2 is a graph displaying the experimental data demonstrating an increase in FGFR3 expression following treatment with BMP-2 over a time span of twenty-four days (open circles=control, closed circles=BMP-2).

FIG. 3 is a graph displaying experimental data from a quantitative PCR assay demonstrating an increase in FGFR3 expression in human FSCs following treatment with BNT-2.

FIG. 4 is a graph displaying experimental data from a quantitative PCR assay demonstrating an increase in FGFR3 expression in human MSCs following treatment with BMP-2.

FIG. 5 is a bar graph displaying experimental data from a quantitative PCR assay demonstrating the relative FGFR3 expression levels in different tissues.

FIG. 6 is a bar graph displaying experimental data from an eNorthem assay demonstrating the relative FGFR3 expression levels in different tissues.

FIG. 7 is a bar graph displaying experimental data demonstrating the effect of FGF-9 on ALPase activity in MSCs (top). Also shown is a bar graph displaying experimental data from a crystal violet proliferation assay demonstrating the effect of FGF-9 on MSC proliferation (bottom).

FIG. 8 is a graph displaying experimental data from a murine calvarial organ culture model demonstrating the effects of FGF-9 on percentage change in weight.

FIG. 9 is a photomicrograph of representative sections of calvaria treated with control media or media containing FGF-9. Sections are stained with H&E and photos were taken using a 10× objective.

FIG. 10 is a graph displaying experimental data from a murine calvarial local injection model demonstrating the effects of FGF-9 on the thickness of calvarial bones.

### DETAILED DESCRIPTION

General Description

The present invention is based in part on identifying genes that are differentially regulated or expressed in bone deposition disorders. Specifically, Fibroblast Growth Factor Receptor-3 (FGFR3) has been identified as being differentially regulated during the maturation of osteoblasts and whose expression can be correlated, for example, with bone deposition disorders such as osteoporosis (including correlation with degrees of severity of the disease). Further, monitoring of expression may be indicative of treatment efficacy. The FGFR3 gene or fragments of this gene, as well as the peptides they encode, can serve as targets for agents that can be used to modulate the activity of FGFR3. For example, agents may be identified which bind to FGFR3 and modulate biological processes associated with bone deposition such as differentiation of stem cells into osteoblasts.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, the term “bone density” refers to the mass or quantity of bone tissue in a certain volume of bone.

As used herein, the term “bone deposition” refers the formation of new bone during osteogenesis.

As used herein, the term “bone resorption” refers to a decrease in bone density and/or mass. Generally, mechanisms of bone resorption include, but are not limited to, secretion of enzymes and/or acids by osteoclasts to facilitate the breakdown of bone.

As used herein, the term “Fibroblast Growth Factor 9” or “FGF9” refers to a growth factor protein which has high binding affinity for FGFR3, substantially lower binding affinity for FGFR2 and no binding affinity for FGFR1 or FGFR4. Examples of FGF9 include, but are not limited to, those disclosed in SEQ ID NO: 4, WO 96/41523 and GenBank Accession No. XM007105.

As used herein, the term “Fibroblast Growth Factor Receptor 3” or “FGFR3” refers to a transmembrane tyrosine linase that has high binding affinity for FGF9. Examples of FGFR3 include, but are not limited to those disclosed in SEQ ID NO: 2, WO 96/41523 and GenBank Accession No. XM017699.

As used herein, the term “osteoporosis” refers to a pathological disorder characterized by a reduction in the amount of bone mass and/or density. Osteoporosis is generally characterized by increased osteoclast activity and/or decreased osteoblast activity.

As used herein, the term “stem cell” or “mesenchymal stem cell” refers to a cell capable of differentiation into an osteoblast cell. These terms are used throughout the specification to indicate that the cell is undifferentiated.

As used herein, the terms “stem cell differentiation” and “osteoblast differentiation” refers to the process in which a stem cell develops specialized functions during maturation into an osteoblast cell.

As used herein, the term “osteoblast” refers to a cell capable of mediating bone deposition. Osteoblasts are derived from mesenchymal stem cells of the bone marrow stroma.

As used herein, the term “osteoclast” refers to a cell capable of mediating bone resorption.

Modulation of FGFR3 Expression

The present inventors have identified FGFR3 as a protein that is associated with mesenchymal stem cell differentiation and subsequent osteoblast activity. Specifically, the expression and activation of FGFR3 in mesenchymal stem cells correlated with the maturation of these cells into osteoblasts and subsequent deposition of bone. The present invention therefore includes methods for modulating FGFR3 expression and/or activity, including methods for modulating FGFR3 signal transduction pathways via downstream membrane and cytoplasmic signaling proteins, to effect mesenchymal stem cell differentiation and osteoblast activity. Such methods will be useful in the treatment of disorders associated with abnormal osteoblast activity. Because osteoblast activity indirectly effects osteoclast activity via a general feedback mechanism, the invention also includes methods for modulating bone resorption associated with osteoclast activity.

Modulation of the FGFR3 gene, gene fragments, or the encoded protein or protein fragments is useful in gene therapy to treat disorders associated with FGFR3 defects. In a preferred embodiment, FGFR3 expression is elevated to increase osteoblast activity in diseases with abnormal bone density. Expression vectors may be used to introduce the FGFR3 gene into a cell as has been demonstrated with constitutively active forms of FGFR3 with any one of the following mutations: lysine to glutamic acid at position 650; arginine to cysteine at position 248; serine to cysteine at position 249; serine to cysteine at position 365; glycine to arginine at position 380; asparagine to lysine or threonine at position 540; and isoleucine to valine at position 538. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termiination region. The transcription cassettes may be introduced into a variety of vectors, e.g., plasmid, retrovirus, lentivirus, adenovirus and the like, where the vectors are able to transiently or stably be maintained in the cells, usually for a period of at least about one day, more usually for a period of at least about several days to several weeks.

The FGFR3 gene or protein may be introduced into tissues or host cells by any number of routes, including viral infection, microinjection, or fusion of vesicles. Jet injection may also be used for intramuscular administration, as described by Furth et al. (1992) Anal. Biochem. 205, 365-368. The DNA may be coated onto gold microparticles, and delivered intradernally by a particle bombardment device, or “gene gun” as described in the literature (see, for example, Tang et al. (1992) Nature 356, 152-154), where gold microprojectiles are coated with FGFR3 DNA, then bombarded into skin cells.

Antisense molecules can be used to down-regulate expression of FGFR3 in cells. The anti-sense reagent may be antisense oligonucleotides, particularly synthetic antisense oligonucleotides having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanusms, e.g., by reducing the amount of mRNA available for translation, through activation of RNAseH or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about seven, usually at least about twelve, and more usually at least about twenty nucleotides in length. Typical antisense oligonucleotides are usually not more than about five-hundred, more usually not more than about fifty, and even more usually not more than about thirty-five nucleotides in length, where the length is governed by efficiency of inhibition, specificity, including absence of cross-reactivity, and the like. It has been found that short oligonucleotides, of from seven to eight bases in length, can be strong and selective inhibitors of gene expression (see Wagner et al. (1996) Nat. Biotech. 14, 840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1996) Nat. Biotech. 14, 840-844). Preferred oligonucleotides are chemically modified from the native phosphodiester structure, in order to increase their-intracellular stability and binding affinity. A number of such modifications have been described in the literature, which alter the chemistry of the backbone, sugars or heterocyclic bases.

As an alternative to anti-sense inhibitors, catalytic nucleic acid compounds, e.g., ribozymes, deoxyribozymes (see, for example, Santoro et al. (1997) Proc. Natl. Acad. Sci. USA 94, 4262-4266), anti-sense conjugates, etc. may be used to inhibit gene expression. Ribozymes may be synthesized in vitro and administered to the patient, or may be encoded on an expression vector, from which the ribozyme is synthesized in the targeted cell (see, for example, WO 95/23225; Beigelman et al. (1995) Nuel. Acids Res. 23, 4434-4442). Examples of oligonucleotides with catalytic activity are described in WO 95/06764.

Screening for Agents which Modulate FGFR3 Expression

Another embodiment of the presenit invention provides methods for identifying agents that modulate the expression of a nucleic acid encoding a FGFR3 protein. Such assays may utilize any available means of monitoring for changes in the expression level of the nucleic acids of the invention. As used herein, an agent is said to modulate the expression of a nucleic acid encoding a FGFR3 protein, if it is capable of up- or down-regulating expression of the nucleic acid in a cell.

In one assay format, cell lines that contain reporter gene fusions between any region of the open reading frame of the FGFR3 gene or fragments thereof under control of the gene's promoter and any assayable fusion partner may be prepared. Numerous assayable fusion partners are known and readily available including the firefly luciferase gene and the gene encoding chloramphenicol acetyltransferase (Alam et al. (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding a FGFR3 protein.

Additional assay formats may be used to monitor the ability of the agent to modulate the expression of a nucleic acid encoding a FGFR3 protein. For instance, mRNA expression may be monitored directly by hybridization to the nucleic acids encoding the FGFR3 gene. Cell lines are exposed to the agent to be tested under appropriate conditions and time and total RNA or mRNA is isolated by standard procedures such those disclosed in Sambrook et al. (1985) Molecular Cloning: A Laboratory Manual, Cold Spring Harbor Laboratory Press.

Probes to detect differences in RNA expression levels between cells exposed to the agent and control cells may be prepared from the nucleic acids encoding the FGFR3 gene. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complimentarily which should exist between two nucleic acid strands in order to form a hybrid. Stringency should be chosen to maximize the difference in stability between the probe:target hybrid and potential probe:non-target hybrids.

Probes may be designed from the nucleic acids encoding the FGFR3 gene through methods known in the art. For instance, the G+C content of the probe and the probe length can affect probe binding to its target sequence. Methods to optimize probe specificity are commonly available in Sambrook et al. (1989) Molecular Cloning: A Laboratory Manual, Cold Spring Harbor Laboratory Press; or Ausubel et al. (1995) Current Protocols in Molecular Biology, Greene Publishing Company.

Hybridization conditions are modified using known methods, such as those described by Sambrook et al. (1989) Molecular Cloning: A Laboratory Manual, Cold Spring Harbor Laboratory Press; or Ausubel et al. (1995) Current Protocols in Molecular Biology, Greene Publishing Company as required for each probe. Hybridization of total cellular RNA or RNA enriched for polyadenylated RNA can be accomplished in any available format. For instance, total cellular RNA or RNA enriched for polyadenylated RNA can be affixed to a solid support and the solid support exposed to at least one probe comprising at least one, or part of one of the sequences encoding the FGFR3 gene under conditions in which the probe will specifically hybridize.

Alternatively, nucleic acid fragments comprising at least one, or part of one of the sequences of the invention can be affixed to a solid support, such as a porous glass wafer. The glass wafer can then be exposed to total cellular RNA or polyadenylated RNA from a sample under conditions in which the affixed sequences will specifically hybridize. Such glass wafers and hybridization methods are widely available, for example, those disclosed in WO 95/11755. By examining for the ability of a given probe to specifically hybridize to an RNA sample from an untreated cell population and from a cell population exposed to the agent, agents which up or down regulate the expression of a nucleic acid (SEQ ID NO: 1) encoding the FGFR3 protein (SEQ ID NO: 2) are identified.

Hybridization for qualitative and quantitative analysis of mRNA may also be carried out by using a RNase Protection Assay (i.e., RPA, see Ma et al. (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes (pH 6.4), 0.4 M NaCl and 1 mM EDTA. The resulting hybrids are then digested in a buffer comprising 40 mg/ml ribonuclease A and 2 mg/ml ribonuclease. After deactivation and extraction of extraneous proteins, the samples are loaded onto urealpolyacrylamide gels for analysis.

In another assay format, agents which effect the expression of the instant gene products, cells or cell lines would first be identified which express said gene products physiologically. Cells and cell lines so identified, such as cells derived from the bone, would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter upstream of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag. Such a process is well known in the art (see Sambrook et al. (1989) Molecular Cloning: A Laboratory Manual, Cold Spring Harbor Laboratory Press).

Cells or cell lines transduced or transfected as outlined above would then be contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides from disrupted cells are fractionated such that a polypeptide fraction is pooled and contacted with an antibody to be further processed by immunological assay (e.g., ELISA, immunoprecipitation or Western blot). The pool of proteins isolated from the “agent contacted” sample will be compared with a control sample where only the excipient is contacted with the cells and an increase or decrease in the immunologically generated signal from the “agent contacted” sample compared to the control will be used to distinguish the effectiveness of the agent.

Methods to Identify Agents that Modulate FGFR3 Activity

The present invention provides methods for identifying agents that modulate at least one activity of a FGFR3 protein. Such methods or assays may utilize any means of monitoring or detecting the desired activity.

In one format, the specific activity of a FGFR3 protein, normalized to a standard unit, between a cell population that has been exposed to the agent to be tested compared to an unexposed control cell population may be assayed. Cell lines or populations are exposed to the agent to be tested under appropriate conditions and time. Cellular lysates may be prepared from the exposed cell line or population and a control, unexposed cell line or population. The cellular lysates are then analyzed with the probe.

Other screening assays may include measuring FGFR3 activity by determining intracellular calcium concentrations. This could be accomplished by screening compounds in cells containing FGFR3, determine calcium content by appropriate method, and then screen compounds in cell line not expressing FGFR3 as a negative control. Compounds which could act through FGFR3 activation would be those increasing calcium in a FGFR3-positive cell line, but not in a FGFR3-negative cell line. Kinase activity assays could also be constructed where cells are stimulated with screening compounds followed by exposure of the cell lysate (or sub-lysate fraction) to a specific FGFR3 kinase substrate to monitor the activation of intrinsic receptor kinase activity. The association of specific binding proteins with FGFR3 could also be used as an indication of receptor activation.

In yet another embodiment, one could test agents to identify which agents bind to FGFR3. Methods of determining binding of a compound to a receptor are well kcnown in the art. Typically, the assays include the steps of incubating a source of the FGFR3 with a labeled compound, known to bind to the receptor, in the presence or absence of a test compound and determining the amount of bound labeled compound. The source of FGFR3 may either be cells expressing FGFR3 or some form of isolated FGFR3 as described herein. The labeled compound can be FGF9 or any FGF9 analog labeled such that it can be measured quantitatively (e.g., ¹²⁵I-labeled, europium labeled, fluorescein labeled, GFP labeled, ³⁵S-methionine labeled). Test compounds that bind to the FGFR3 cause a reduction in the amount of labeled ligand bound to the receptor, thereby reducing the signal level compared to that from control samples (absence of test compound).

Antibody probes can be prepared by immunizing suitable mammalian hosts utilizing appropriate immunization protocols using the FGFR3 protein or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) Biotechnology 24, 524-526 or modifications which effect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies may be recovered from the culture supernatant or from the ascites supernatant. The intact anti-FGFR3 antibodies or fragments thereof which contain the immunologically significant portion can be used as e.g., antagonists of binding between FGF9 (ligand) (SEQ ID NO: 4) and FGFR3, or alternatively as a FGFR3 agonists. Use of immunologically reactive fragments, such as Fab or Fab′ fragments, is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin.

Antibody regions that bind specifically to the desired regions of the protein can also be produced in the context of chimeras with multiple species origin, for instance, humanized antibodies. The antibody can therefore be a humanized antibody or human a antibody, as described in U.S. Pat. No. 5,585,089 or Riechmann et al. (1988) Nature 332, 323-327.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to the extracellular domain of an FGFR3 which interacts with FGF9. Alternatively, it can be a fragment of the extracellular domain.

The agents of the present invention can be, as examples, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies or fragments thereof that bind to a FGFR3 or FGF9 protein. Antibody agents can be obtained by immunization of suitable mammalian subjects with peptides, containing as antigenic regions, those portions of the protein intended to be targeted by the antibodies.

In yet another class of agents, the present invention includes peptide mimetics which mimic the three-dimensional structure of FGF9 and bind to FGFR3. Such peptide mimetics may have significant advantages over naturally-occurring peptides, including, for example: more economical production, greater chemical stability, enhanced pharmacological properties (half-life, absorption, potency, efficacy, etc.), altered-specificity (e.g., a broad-spectrum of biological activities), reduced antigenicity and others.

In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

In another form, peptide analogs are commonly used in the pharmaceutical industry as non-peptide drugs with properties analogous to those of the template peptide. These types of non-peptide compounds are also referred to as peptide mimetics or peptidomnimetics (Fauchere (1986) Adv. Drug Res. 15, 29-69; Veber & Freidinger (1985) Trends Neurosci. 8, 392-396; Evans et al. (1987) J. Med. Chem. 30, 1229-1239 which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), such as the binding domain of FGF9, but have one or more peptide linkages optionally replaced by a linkage by methods known in the art.

Labeling of peptide mimetics usually involves covalent attachment of one or more labels, directly or through a spacer (e.g. an amide group), to non-interfering position(s) on the peptide mimetic that are predicted by quantitative structure-activity data and molecular modeling. Such non-interfering positions generally are positions that do not form direct contacts with the macromolecule(s) (e.g., are not contact points in FGF9-FGFR3 complexes) to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of FGF9 to FGFR3. Approaches that can be used include the yeast two hybrid method (see Chien et al. (1991) Proc. Natl. Acad. Sci. USA 88, 9578-9582) and using the phage display method. The two hybrid method detects protein-protein interactions in yeast (Fields et al. (1989) Nature 340, 245-246). The phage display method detects the interaction between an immobilized protein and a protein that is expressed on the surface of phages such as lambda and M13 (Amberg et al. (1993) Strategies 6, 2-4; Hogrefe et al. (1993) Gene 128, 119-126). These methods allow positive and negative selection for protein-protein interactions and the identification of the sequences that determine these interactions.

An additional class of agents that could be screened and used to activate the FGFR3 are agentsthat can either directly or indirectly activate the kinase domain of thus receptor and influence mesenchymal stem cell differentiation into osteoblasts and/or promote osteoblast activity. Such examples of these kinase effectors have been previously described (see, for example, Salituro et al. (2001) Recent Prog. Horm. Res. 56, 107-126; Zhang et al. (1999) Science 284, 886-887).

Diagnostic Uses for FGFR3

As described above, FGFR3 expression may be used as a diagnostic marker for the prediction or identification of the differentiation state of a sample comprising precursor stem cells. In some embodiments, the tissue sample is a bone biopsy. For instance, a tissue sample may be assayed by any of the methods described above, and FGFR3 expression levels may be compared to the expression levels found in undifferentiated precursor stem cells and/or precursor stem cells induced to differentiate into osteoblasts and/or precursor stem cells induced to differentiate into a cell type other than an osteoblast. Such methods may be used to diagnose or identify conditions characterized by abnormal bone deposition, reabsorption and/or abnormal rates of osteoblast differentiation.

Those skilled in the art will appreciate that a wide variety of conditions are associated with abnormal bone deposition or loss. Such conditions include, but are not limited to, osteoporosis, osteopenia, osteodystrophy, and various other osteopathic conditions. The methods of the present invention will be particularly useful in diagnosing or monitoring the treatment of conditions such as postmenopausal osteoporosis (PMO), glucocorticoid-induced osteoporosis (GIO), and male osteoporosis. Agents which modulate FGFR3 expression will be useful in treatment of these conditions.

In some preferred embodiments, the present invention may be used to diagnose and/or monitor the treatment of drug-induced abnormalities in bone formation or loss. For example, at present a combination of cyclosporine with prednisone is given to patients who have received an organ transplant in order to suppress tissue rejection. The combination causes rapid bone loss in a manner different than that observed with prednisone alone (such as elevated level of serum osteocalcin and vitamnin D in patients treated with cyclosporine but not in patients treated with prednisone). Other drugs are also known to effect bone formation or loss. The anticonvulsant drugs diphenylhydantoin, phenobarbital and carbamazepine, and combination of these drugs, cause alterations in calcium metabolism. A decrease in bone density is observed in patients taking anticonvulsant drugs. Although heparin is an effective therapy for thromboembolic disorders, increased incidences of osteoporotic fractures have been reported in patients with heparin therapy hence the present invention will be useful to monitor patients undergoing heparin treatment.

Other embodiments of the present invention allow the diagnosis and/or monitoring of the treatment of other conditions that involve altered bone metabolism. For example, idiopathic juvenile osteoporosis (IJO) is a generalized decrease in mineralized bone in the absence of rickets or excessive bone resorption and typically occurs in children before the onset of puberty. In addition, thyroid diseases have been linked to bone loss. A decrease in bone mass has been shown in patients with thyrotoxicosis causing these individuals to be at increased risk of having fractures. These individuals also sustain fractures at an earlier age than individuals who have never been thyrotoxic.

Another situation in which the present invention will be useful is the diagnosis and/or monitoring of the treatment of skeletal disease linked to breast cancer. Breast cancer frequently metastasizes to the skeleton and about 70% of patients with advanced cancer develop symptomatic skeletal disease. Moreover, the anticancer treatments presently in use have been shown to lead to early menopause and bone loss when given to premenopausal women.

The present invention will be useful in diagnosing and/or monitoring the treatment of chronic anemia associated with abnormal bone formation or loss. Homozygous beta-thalassemia is usually described as an example of chronic anemia predisposing to osteoporosis. Patients with thalassemia have expansion of bone marrow space with thinning of the adjacent trabeculae.

Other conditions in which the present invention will find application are: Fanconi syndrome where osteomalacia is a common feature; fibrous dysplasia, McCune-Albright syndrome refers to patients with fibrous dysplasia with a sporadic, developmental disorder characterized by a unifocal or multifdcal expanding fibrous lesion of bone-forming mesenchyme that often results in pain, fracture or deformity; osteogenesis imperfecta (OI, also called brittle bone disease) is associated with recurrent fractures and skeletal deformity, various skeletal dysplasias i.e., osteochondroplasia which is characterized by abnormal development of cartilage and/or bone and other diseases such as achodroplasia, mucopolysacchaidoses, dysostosis and ischemic bone diseases.

The present invention will be particularly useful by providing a marker which may be used as a marker of bone turnover to determine osteoporosis. The present invention may also be used in vitro in assays or treatments as a marker of osteoblast differentiation and proliferation.

Methods of Treatment Associated with Modulation of FGFR3 Expression

As provided in the Examples, the FGFR3 proteins and nucleic acids are expressed on osteoblasts derived from mesenchymal stem cells. Agents that modulate or up- or down-regulate the expression of the FGFR3 protein or agents such as agonists or antagonists of at least one activity of the FGFR3 protein may be used to modulate biological and pathologic processes associated with the protein's function and activity. The invention is particularly useful in the treatment of human subjects.

Pathological processes refer to a category of biological processes which produce a deleterious effect. For example, expression of FGFR3 is associated with differentiation of stem cells into osteoblasts under normal conditions but in a disease state, the necessary level of FGFR3 expression may not be present. Such diseases include, but are not limited to, diseases caused by an abnormal rate of osteoblast formation and subsequent activity. Decreased osteoblast activity can lead to a decrease in bone deposition with a concurrent increased osteoclast activity resulting in abnormal increase in bone resorption ultimately leading to decreased bone density.

As discussed above, those skilled in the art will appreciate that a wide variety of conditions are associated with an abnormal rate of osteoblast formation leading to abnormal bone deposition or loss. Such conditions include, but are not limited to, osteoporosis, osteopenia, osteodystrophy, and various other osteopathic conditions. The methods of the present invention will be particularly useful in the treatment of conditions such as postmenopausal osteoporosis (PMO), glucocorticoid-induced osteoporosis (GIO), and male osteoporosis. Agents which modulate FGFR3 expression will be useful in treatment of these conditions.

Osteoporosis is an example of one such disease characterized by abnormal bone density. As used herein, an agent is said to modulate a pathological process when the agent reduces the degree or severity of the process. For instance, a bone density disorder may be prevented or disease progression modulated by the administration of agents which reduce, promote or modulate in some way the expression or at least one activity of FGFR3. For osteoporosis, the therapeutic strategy comprises a treatment with the agent until normal bone mass compared to appropriate control groups is restored. Bone mass can be assessed by determining bone mineral density. Then the treatment can be switched to established regimens for the prevention of bone loss to avoid potential side effects of overshooting bone formation.

Other embodiments of the present invention allow for the treatment of other conditions that involve altered bone metabolism associated with osteoblast activity. For example, idiopathic juvenile osteoporosis (IJO) is a generalized decrease in mineralized bone in the absence of rickets or excessive bone resorption and typically occurs in children before the onset of puberty. In addition, thyroid diseases have been linked to bone loss. A decrease in bone mass has been shown in patients with thyrotoxicosis causing these individuals to be at increased risk of having fractures. These individuals also sustain fractures at an earlier age than individuals who have never been thyrotoxic.

The present invention will be useful in the treatment of abnormal bone formation or loss associated with chronic anemia. Homozygous beta-thalassemia is usually described as an example of chronic anemia predisposing to osteoporosis. Patients with thalassemia have expansion of bone marrow space with thinning of the adjacent trabeculae.

Other conditions in which the present invention will find therapeutic application are: Fanconi syndrome where osteomalacia is a common feature; fibrous dysplasia, McCune-Albright syndrome refers to patients with fibrous dysplasia with a sporadic, developmental disorder characterized by a unifocal or multifocal expanding fibrous lesion of bone-forming mesenchyme that often results in pain, fracture or deformity; osteogenesis imperfecta (OI, also called brittle bone disease) is associated with recurrent fractures and skeletal deformity, various skeletal dysplasias i.e., osteochondroplasia which is characterized by abnormal development of cartilage and/or bone and other diseases such as achodroplasia, mucopolysacchaidoses, dysostosis and ischemic bone diseases.

In one example, administration of FGF9-like peptide agents can be used to treat a bone density disorder associated with the FGFR3 protein. In another example, administration of soluble FGFR3 protein can be used to treat a bone density disorder associated with FGFR3 expression. Soluble receptors have been used to bind cytokines or other ligands to regulate their function (Thomson (1998) Cytokine Handbook, Academic Press). A soluble receptor occurs in solution, or outside of the membrane. Soluble receptors may occur because the segment of the molecule which spans or associates with the membrane is absent. This segment is commonly referred to in the art as the transmembrane domain of the gene, or membrane binding segment of the protein. Thus, in some embodiments of the invention, a soluble receptor includes a fragment or an analog of a membrane bound receptor. Preferably, the fragment contains at least six, e.g., ten, fifteen, twenty, twenty-five, thirty, forty, fifty, sixty or seventy amino acids, provided it retains its desired activity.

In other embodiments of the invention, the structure of the segment that associates with the membrane is modified (e.g., DNA sequence polymorphism or mutation in the gene) so the receptor is not inserted into the membrane, or the receptor is inserted, but is not retained within the membrane. Thus, a soluble receptor, in contrast to the corresponding membrane bound form, differs in one or more segments of the gene or receptor protein that are important to its association with the membrane.

The agents of the present invention can be provided alone, or in combination, or in sequential combination with other agents that modulate a particular pathological process. As used herein, two agents are said to be administered in combination when the two agents are administered simultaneously or are administered independently in a fashion such that the agents will act at the same time. For example, the agents of the invention can be used in combination with estrogen replacement therapy in postmenopausal osteoporosis.

The agents of the present invention can be administered via parenteral, subcutaneous, intravenous, intramuscular, intraperitoneal, transdermal, or buccal routes. For example, an agent may be administered locally to a site of injury via microinfusion. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents which modulate expression or at least one activity of a FGFR3 protein. While individual needs vary, determination of optimal ranges of effective amounts of each component is within the skill of the art. Typical dosages comprise 1 pg/kg to 100 mg/kg body weight. The preferred dosages for systemic administration comprise 100 ng/kg to 100 mg/kg body weight. The preferred dosages for direct administration to a site via microinfusion comprise 1 ng/kg to 1 mg/kg body weight.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient. Suitable formulations for oral administration include hard or soft gelatin capsules, pills, tablets, including coated tablets, elixirs, suspensions, syrups or inhalations and controlled release forms thereof.

In practicing the methods of this invention, the agents of this invention may be used alone or in combination, or in combination with other therapeutic or diagnostic agents. In certain preferred embodiments, the compounds of this invention may be co-administered along with other compounds typically prescribed for these conditions according to generally accepted medical practice, such as anti-inflammatory agents, anticoagulants, antithrombotics, including platelet aggregation inhibitors, tissue plasminogen activators, urokinase, prourokinase, streptokinase, aspirin and heparin. The compounds of this invention can be utilized in vivo, ordinarily in mammals, such as humans, sheep, horses, cattle, pigs, dogs, cats, rats and mice, or ini vitro.

Prognostic Uses for FGFR3

As described above, FGFR3 gene and FGFR3 gene expression may also be used as a marker for the monitoring of disease progression, such as osteoporosis. For instance, a tissue sample may be assayed by any of the methods described above, and the expression levels for the FGFR3 gene may be compared to the expression levels found in undifferentiated precursor stem cells and/or precursor stem cells induced to differentiate into osteoblasts and/or precursor stem cells induced to differentiate into a cell type other than an osteoblast and/or osteoblasts.

FGFR3 expression or activity may also be used to track or predict the progress or efficacy of a treatment regime in a patient. For instance, a patient's progress or response to a given drug may be monitored by measuring FGFR3 gene expression in a tissue or cell sample after treatment or administration of the drug. FGFR3 gene expression in the post-treatment sample may then be compared to gene expression from undifferentiated precursor stem cells and/or precursor stem cells induced to differentiate into osteoblasts and/or precursor stem cells induced to differentiate into a cell type other than an osteoblast and/or osteoblasts and/or from tissue or cells from the same patient before treatment.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1

Up-regulation of FGFR3 Expression in hFSC

Human Fetal Stromal Cells (HFSC) were isolated from the bone marrow of a twenty-week human embryo. hFSCs are derived from a primary culture and represent a heterogeneous population of osteoprogenitor cells. hFSCs exhibit a high replicative capacity, with a doubling time of approximately twenty hours. hFSCs retain a spindle-shaped morphology and have a uniform attachment throughout subcultivation. hFSCs can be sub-cultured up to twelve passages while retaining both proliferative and osteogenic capability.

hFSCs used for READS analysis or Q-PCR were cultured in Dulbecco's Modified Eagle Medium (DMEM)-high glucose or DMEM-low glucose+10% fetal bovine serum, respectively, at 37° C. in a humidified atmosphere containing 95% air and 5% carbon dioxide in the absence and presence of the indicated treatment. RNA was extracted from the cells at thirty minutes, three hours, six hours, twelve hours, twenty-four hours, forty-eight hours, three days, six days, twelve days and twenty-four days. When indicated, cells were contacted with either bone morphogenic protein-2 (13MP-2) at 300 ng/ml or transforming growth factor beta (TGFO) at 1 ng/ml. Cells were incubated for the period of time indicated and harvested.

Total cellular RNA was prepared from the human fetal stromal cells described above. Synthesis of cDNA was performed as previously described in WO 97/05286 and in Prashar et al. (1996) Proc. Natl. Acad. Sci. USA 93, 659-663. Briefly, cDNA was synthesized according to the protocol described in the Gibco-BRL kit for cDNA synthesis. The reaction mixture for first-strand synthesis included 0.006 mg of total RNA, and 200 ng of a mixture of one-base anchored oligo(dT) primers with all three possible anchored bases (acgtaatacgactcactatagggcgaattgggtcgact₁₇n1 wherein n1=a, c or g) (SEQ ID NO: 5) along with other components for first-strand synthesis reaction except reverse transcriptase. This mixture was incubated at 65° C. for five minutes, chilled on ice and the process repeated.

Alternatively, the reaction mixture may include 0.010 mg of total RNA, and 2 pmol of one of the two base anchored oligo(dT) primers annealed such as RP5 (ctctcaaggatcttaccgctt₁₈at) (SEQ ID NO: 6), RP6 (taataccgcgccacatagcat₁₈cg) (SEQ ID NO: 7) or RP92 (cagggtagacgacgctacgct₁₈ga) (SEQ ID NO: 8) along with other components for first-strand synthesis reaction except reverse transcriptase. This mixture was then layered with mineral oil and incubated at 65° C. for seven minutes followed by 50° C. for another seven minutes. At this stage, 0.002 ml of Superscripte reverse transcriptase (Gibco-BRL) (200 units per microliter) was added quickly and mixed, and the reaction continued for one hour at 45-50° C. Second-strand synthesis was performed at 16° C. for two hours. At the end of the reaction, the cDNA were precipitated with ethanol and the yield of cDNA was calculated. In these experiments, 200 ng of cDNA was obtained from 0.010 mg of total RNA. The adapter oligonucleotide sequences were A1 (tagcgtccggcgcagcgacggccag) (SEQ ID NO: 9) and A2 (gatcctggccgtcggctgtctgtcggcgc) (SEQ ID NO: 10).

One microgram of oligonucleotide A2 was first phosphorylated at the 5′ end using T4 polynucleotide k:inase (PNK). After phosphorylation, PNK was heated denatured and 0.001 mg of the oligonucleotide A1 was added along with 10× annealing buffer (1 M NaCl/100 mM Tris—HCl (pH 8.0)/10 mM EDTA (pH 8.0)) in a final volume of 0.020 ml. This mixture was then heated at 65° C. for ten minutes followed by slow cooling to room temperature for thirty minutes, resulting in formation of the Y adapter at a final concentration of 100 ng per microliter. About 20 ng of the cDNA was digested with four units of BglII in a final volume of 0.01 ml for thirty minutes at 37° C. Two microliters (4 ng of digested cDNA) of this reaction mixture was then used for ligation to 100 ng (fifty-fold) of the Y-shaped adapter in a final volume of 0.005 ml for sixteen hours at 15° C. After ligation, the reaction mixture was diluted with water to a final volume of 0.080 ml (adapter ligated cDNA concentration, 0.05 ng/ml) and heated at 65° C. for ten minutes to denature T4 DNA ligase and 0.002 ml aliquots (with 100 pg of cDNA) were used for PCR.

The following sets of primers were used for PCR amplification of the adapter ligated 3′-end cDNA: tgaagccgagacgtcggteg(t)₁₈ n1, n2 (SEQ ID NO: 11) (wherein n1, n2=aa, ac, ag, at, ca, cc, cg, ct, ga, gc, gg and gt) as the 3′ primer with A1 as the 5′ primer or alternatively P5, RP6 or RP92 used as 3′ primers with primer A1.1 serving as the 5′ primer. To detect the PCR products on the display gel, 24 pmol of oligonucleotide A1 or A11 was 5′-end labeled using 0.015 ml of gamma-[³²P]ATP (Amersham; 3000 Ci/mmol) and PNK in a final volume of 0.020 ml for thirty minutes at 37° C. After heat denaturing PNK at 65° C. for twenty minutes, the labeled oligonucleotide was diluted to a final concentration of 0.002 mM in 0.080 ml with unlabeled oligonucleotide A11. The PCR mixture (0.020 ml) consisted of 0.002 ml (100 pg) of the template, 0.002 ml of 10× PCR buffer (100 mM Tris—HCl (pH 8.3)/500 mM KCl), 0.002 ml of 15 mM magnesium chloride to yield 1.5 mM final magnesium concentration optimum in the reaction mixture, 0.20 mM dNTPs, 200 nM each 5′ and 3′ PCR primers, and one unit of Amplitaq Gold® DNA polymerase.

Primers and dNTPs were added after preheating the reaction mixture containing the rest of the components at 85° C. This “hot start” PCR was done to avoid amplification artifacts arising out of arbitrary annealing of PCR primers at lower temperature during transition from room temperature to 94° C. in the first PCR cycle. PCR consisted of five cycles of 94° C. for thirty seconds, 55° C. for two minutes and 72° C. for sixty seconds followed by twenty-five cycles of 94° C. for thirty seconds, 60° C. for two minutes, and 72° C. for sixty seconds. A higher number of cycles resulted in smeary gel patterns. PCR products (0.0025 ml) were analyzed on 6% polyacrylamide sequencing gel. For double or multiple digestion following adapter ligation, 0.0132 ml of the ligated cDNA sample was digested with a secondary restriction enzymes in a final volume of 0.020 ml. From this solution, 0.003 ml was used as template for PCR. This template volume of carried 100 pg of the cDNA and 10 mM magnesium chloride (from the 10× enzyme buffer), which diluted to the optimum of 1.5 mM in the final PCR volume of 0.020 ml. Since magnesium comes from the restriction enzyme buffer, it was not included in the reaction mixture when amplifying secondarily cut cDNA.

Individual cDNA fragments corresponding to FGFR3 mRNA species were separated by denaturing polyacrylamide gel electrophoresis and visualized by autoradiography. Bands identified as having different expression levels in treated versus untreated human fetal stromal cells were extracted from the display gels as described by Liang et al. (1995) Curr. Opin. Immunol. 7, 274-280), reamplified using the 5′ and 3′ primers, and subdloned into pCR-Script with high efficiency using the PCR-Script® cloning kit (Stratagene). Plasmids were sequenced by cycle sequencing on an ABI automated sequencer. Alternatively, bands were extracted (cored) from the display gels, PCR amplified and sequenced directly without subdloning.

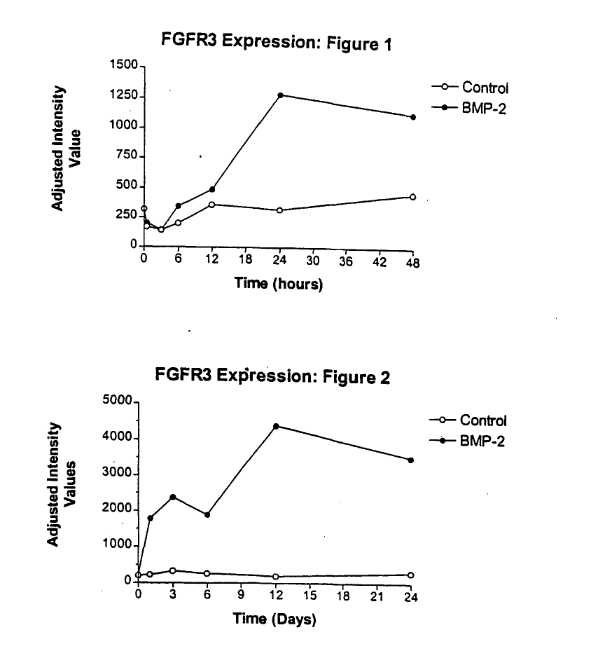

FIG. 1 presents a graphic depiction of the expression level of FGFR3 whose expression pattern was found to be dependent upon the activation state of the precursor stem cells. This figure represents the data obtained from READS gel analysis of the mRNA expression data from HFSC. READS analysis (as described above) was performed on total RNA samples isolated from hFSC that were treated with either TGFβ (1 ng/ml of culture media) or BMP-2 (300 ng/ml of culture media) for up to twenty-four days. Time points were selected at one, three, six, twelve and twenty-four days post-initial treatment. Control cells received media only with no added osteogenic agent. Subsequent to READS gel analysis, the images of each gel were converted into electronic format and the intensities of each band of interest were calculated relative to the background autoradiographjc intensity of each gel image. The corrected values are termed adjusted intensity values, which were plotted on the y-axis versus the time course of the experiment.

### Example 2

Up-Regulation of FGFR3 Expression in hMSC

Both human fetal stromal cells (hFSC) and hMSC were used for this study as in the READS experiments. Briefly, PCR primers and TaqMan probes were designed using the DNA sequences provided by sequence analysis of the FGFR3 nucleotide sequence. Experimental conditions were as follows: HFSC were cultured in vitro and were left untreated for up to twenty-four days, or were treated with the osteogenic agents TGFβ (1 ng/ml of culture media) or BMP-2 (300 ng/ml of culture media) for the same time period. Cells in each of the treatment groups were harvested at one, three, six, twelve and twenty-four hours after addition of TGFβ or BMP-2. Total RNA was isolated from the cells using Trizol® and the RNA was quantitated using a spectrophotometer set at A₂₆₀. Ten ng of total RNA was assayed in duplicate using the TaqMan® assay (Perkin-Elmer) in biplex format where each target gene in each RNA sample was assayed versus a reference mRNA which was shown previously to be constitutively expressed and not regulated by any of the osteogenic treatments. The Ct values of the target and reference gene were analyzed and the delta Ct values were calculated for each RNA sample. Fold change (expressed as relative expression) was plotted versus the time course of the experiment. Expression was relative to the delta Ct value (Target Ct minus Reference Ct) for t=0 which was set to a value of 1.0.

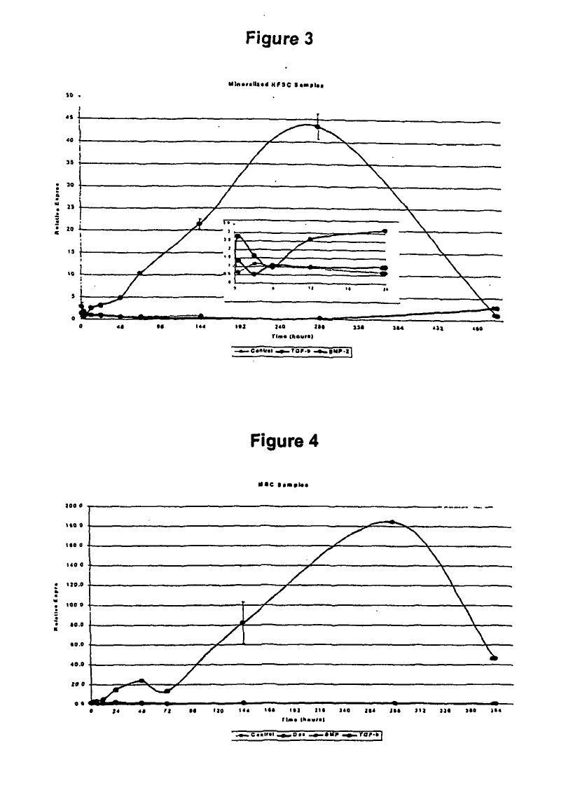

FIG. 3 shows the expression level of the RNA related to FGFR3 mRNA as a function of time in the absence (control-open circles) and in the presence (BMP-2-closed squares) of 300 ng/ml BMP-2 or in the presence (TGFβ-closed circles) of 1 ng/ml TGFT.

### Example 3

FGFR3 Expression in Human Tissues

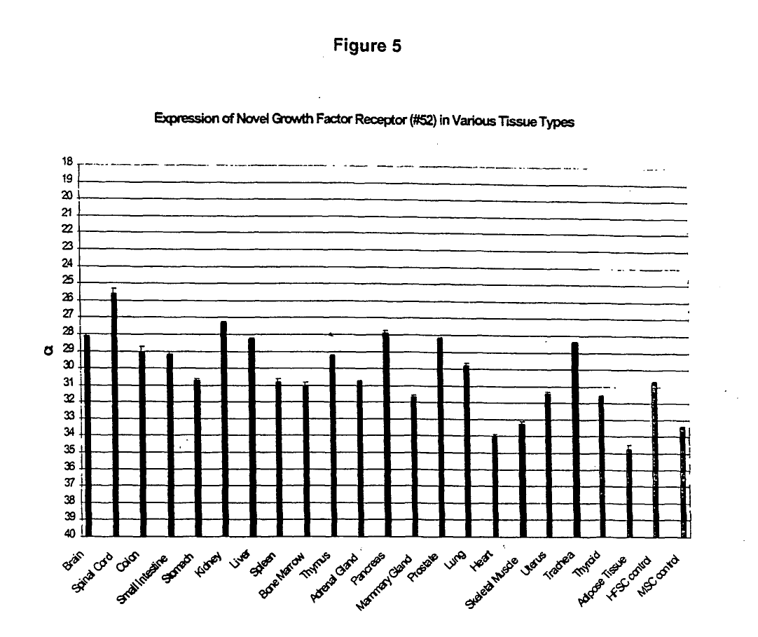

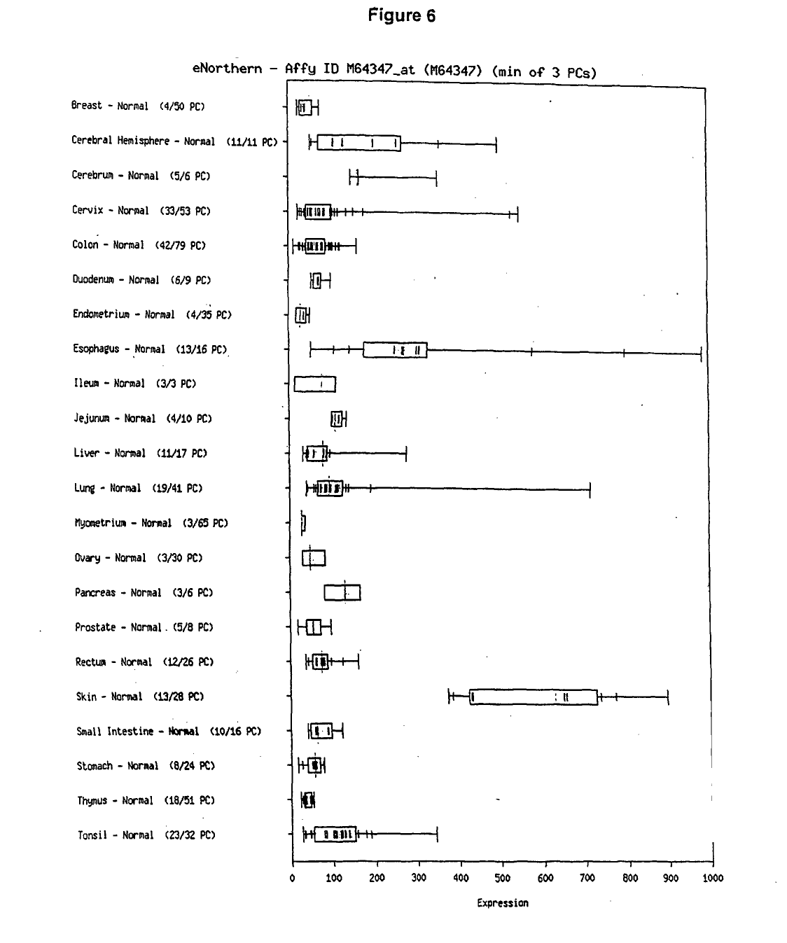

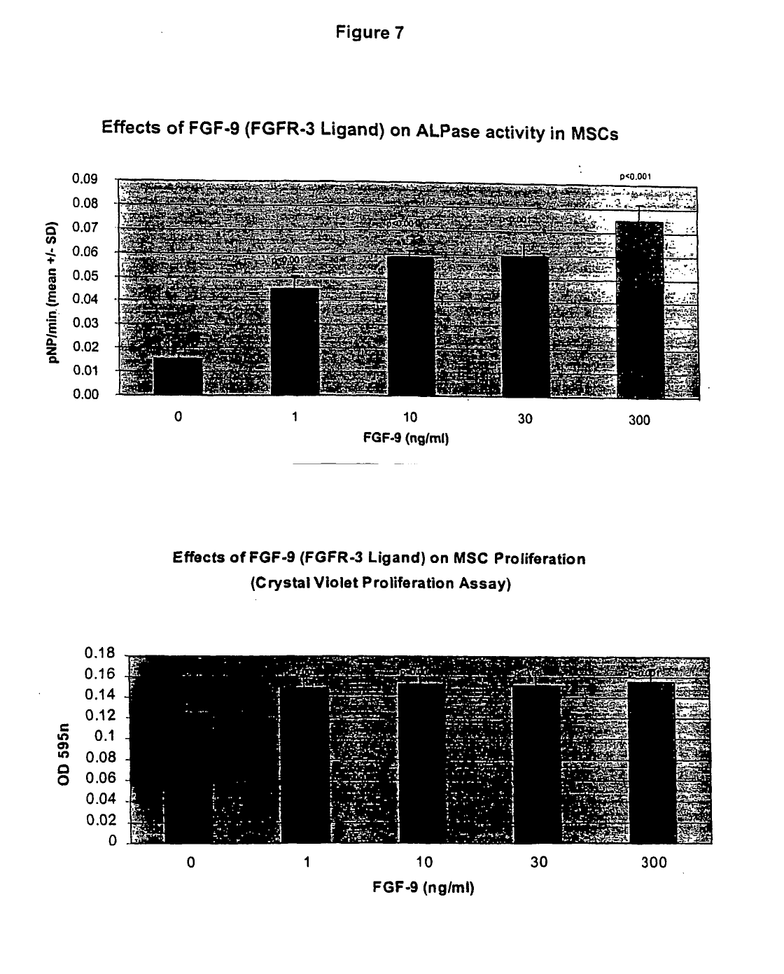

The tissue distribution of mRNA encoding the FGFR3 gene was analyzed by quantitative PCR expression analysis of RNA isolated from various tissues. RNA was isolated from human kidney, adrenal gland, pancreas, salivary gland, liver, prostate, thyroid, cerebellum, fetal brain, placenta, spinal cord, stomach, small intestine, bone marrow, thymus, spleen, heart, lung, testes, uterus, mammary gland and trachea using standard procedures. PCR expression analysis was also performed using primers specific for FGFR3 (SEQ ID NO: 12 & SEQ ID NO: 13) as well as a probe derived from SEQ ID NO: 14 using AmpliTaqo PCR amplification kits (Perkin Elmer). The presence of variable levels of FGFR3 mRNA was detected in several tissues other than hFSC and hMSC (FIG. 5). FGFR3 mRNA expression was most abundant in the spinal chord, kidney and pancreas. Lower, but detectable levels, were observed in all other tissues tested.

### Example 4

eNorthem Analysis

Tissues were isolated from normal human subjects and RNA for Affymetrix GeneChip microarray application was prepared with minor modifications following the protocols set forth by the manufacturer. Frozen tissues were ground to a fine powder using a Spex Certiprep 6800 Frezer Mill. Total RNA was extracted with Trizol (Invitrogen) utilizing the manufacturer's protocol. Double-stranded cDNA was generated from the RNA using the SuperScript Choice-System (Invitrogen). First strand synthesis was primed with a T7-(dT24) oligonucleotide. The cDNA was phoneol-chloroform extracted and ethanol precipitated to a final concentration of 1.0 mg/ml. From 0.002 mg of cDNA, cRNA was synthesized using the T7 MegaScripte in vitro Transcription Kit (Ambion). To biotin label the cRNA, nucleotides Bio-11-CTP and Bio-16-CTP (Enzo Diagnostics) were included in the reaction. Following a 37° C. incubation for six hours, impurities were removed from the labeled cRNA by using the RNAeasy® Mini Kit column and protocol (Qiagen). The cRNA was fragmented for thirty-five minutes at 94° C. according to the manufacturer's protocol and 0.055 mg of fragmented cRNA was hybridized on the 60K GeneChip set for twenty-four hours in a 45° C. hybridization oven set at 60 rpm. The chips were washed and stained with Streptavidin Phycoerythrin (SAPE; Molecular Probes) in an Affyinetrix fluidics station. To amplify the staining, SAPE solution was added twice with an anti-streptavidin biotinylated antibody (Vector Labs) staining set in between the addition of the solution. Hybridization to the probe arrays was detected by fluorometric scanning (BP Gene Array Scanner). Data was analyzed using Affymetrix GeneChip (v 3.0) data mining software.

### Example 5

FGFR3-Mediated ALPase Activity in MSC

Mesenchymal stem cells were plated into 96 well treated tissue culture plates at a seeding density of 10,000 cells per well. Cells were subsequently cultured until confluent and then treated with the appropriate concentration of FGF9 in the presence of heparin (0.002 mg/ml). After three days, media was replaced with fresh media containing the appropriate additions and cultured for another three days. Alkaline phosphatase enzyme activity of the cell layer was measured by rinsing cells twice with phosphate buffer saline solution followed by incubation with 5 mM p-nitrophenyl phosphate substrate in 50 mM glycine, 1 mM magnesium chloride (pH 10.5) at room temperature for twenty minutes. Absorbance of the final product (p-nitrophenol, a yellow product) was measured at 405 nm using a microplate reader. The amount of p-nitrophenol produced in each sample was calculated using a standard curve run in parallel. Alkaline phosphatase activity was expressed as p-nitrophenol produced per minute per well.

### Example 6

FGFR3-Mediated MSC Proliferation

Mesenchymal stem cells were plated into 96 well tissue culture plates at a seeding density of 1000 cells per well. Cells were subsequently cultured for twenty hours and then treated with the appropriate concentration of FGF9 in the presence of heparin (0.002 mg/ml). After three days in culture, the cells were washed twice with phosphate buffer saline solution, fixed with 15 gluteraldehyde (v/v) for fifteen minutes, rinsed twice with deionized water and then air dried. Cultures were then stained with 0.1% (w/v) crystal violet in water for thirty minutes. After washing, the crystal violet was extracted from the cells using 1% (v/v) Triton x-100 in water. Absorbance of the extracted samples was measured at 595 nm using a microplate reader.

### Example 7

Mouse Calvarial Organ Culture Model (MOC) Assay

Calvarial bones were dissected from three to five day old CDl mice. Calvaria were placed in a petri dish containing BGJb tissue culture media (Sigma) supplemented with bovine serum albumin, sodium bicarbonate, penicllin and streptomyocin (pH 7.1). Calvaria were removed from the petri dish and excess media removed from the calvaria by blotting with sterile gauze. The weight of each calvaria was then determined.

Calvaria were then transferred to twelve well plates (one per well), concave side down, containing one ml of media per well. Calvaria were then incubated at 37° C. for twenty-four hours on a rocking platform at approximately 150 rpm. Media was then removed from the wells and replenished with fresh media containing test agents or control. Calvaria were then incubated for another three days at 37° C. on a rocking platform at approximately 150 rpm. Media was replaced every three days. On day seven, calvaria were removed, blotted dry using sterile gauze and weighed. Calvaria were then placed in vials containing 40% ethanol for twenty-four hours and then transfered to vials containing 70% ethanol.

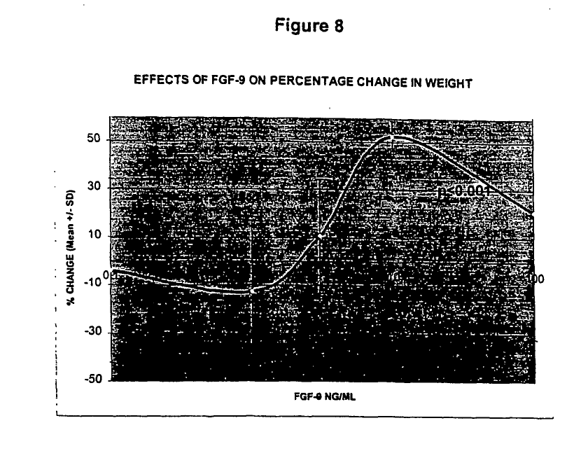

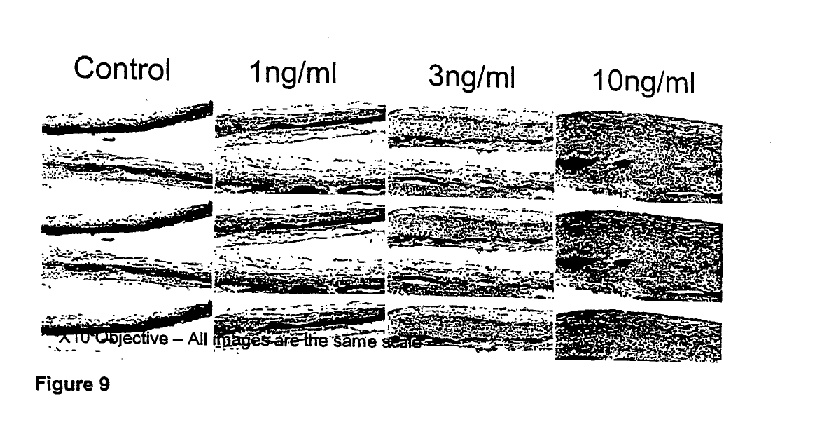

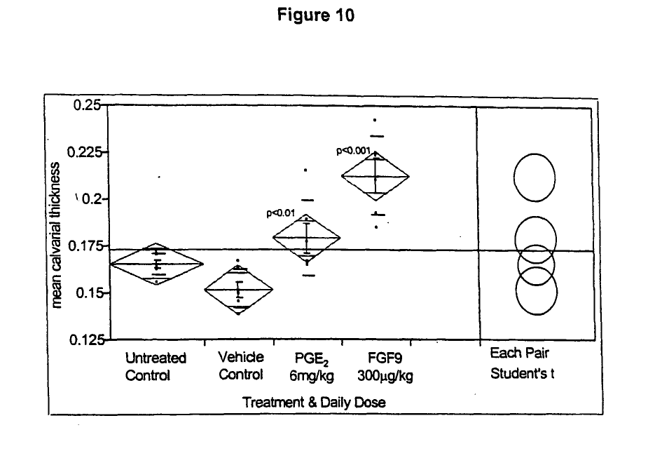

Each calvaria sample taken for histology was notched on the opposite side of the sagittal suture for orientation. Each calvaria was placed in cassettes, embedded in paraffin, cut at four micron thickness starting 800 microns lateral to the sagittal suture, and stained with hematoxylin and eosin (H&E). New and old bone in calvarial sections was identified by its differential color intensity obtained with H&E staining. Their cubodial morphology and purple cytoplasmic staining was used to identify osteoblasts. The effect of FGF9 on calvarial weight measurements is shown in FIG. 8. From the data it is evident that 10 ng/ml FGF9 causes a significant increase in calvarial weight. To determine whether this increase in weight was indicative of an increase in new bone formation, H&E stained histology sections of calvaria were examined. FIG. 10 shows representative sections of calvaria treated with control media or media containing various dilutions of FGF9. Sections are stained with H&E and photos were taken using a 10× objective. All photomicrographs are displayed in the same scale. The control sections clearly demonstrate a layer of old bone (stained dark purple) in the center of the section, surrounded on both sides by a thin layer of osteoid new bone (stained light pink). The FGF9 sections can clearly be seen to be thicker with regards to bone. It is also apparent that the section contains much less old bone, presumably due to an increase in bone turnover and consequently resorption. However a large increase in new bone formation can be seen surrounding the remains of the old bone. In addition to a large increase in bone formation, an increase in cell number can also be seen with large number of cells present on the surface of the new bone. In conclusion there is a significant increase in bone formation in FGF9 treated calvaria as demonstrated by both weight data and histology at doses as low as 1.0 ng/ml.

### Example 8

Mouse Calvarial Local Injection Model

Male Swiss Webster white mice were received at four weeks of age and allowed to acclimate for five days. The mice were injected subcutaneously over the right side of the calvaria for five days with the appropriate factor dilution. Dosing consisted of an injection administered once daily for five consecutive days. The injection site was prepared with a 70% isopropyl wipe and the injection was administered using a Hamilton syringe (100 μl) and a 27-gauge needle (Becton-Dickinson). Dosing solutions were prepared so that 20 μl was administered per animal. Following treatment the mice were allowed to rest for two weeks prior to euthanasia.

At necropsy, the calvaria were removed, cleaned of soft tissue, and fixed in 70% ethanol. The calvaria were examined for any damage associated with scraping of the periosteum with the needle during treatment. The intact calvaria were oriented along the antero-posterior axis with the occipital region resting on the bottom of a 13.00 mm diameter plastic holder tube. A sponge moistened with 70% ethanol was used to secure the calvaria in place in the tube. The sutures on the calvaria were positioned toward the beam to allow a frontal scout view. In the scout view, the reference line was positioned so that the field of view (FOV) included a 3.05 mm region below the coronal suture of the entire calvaria. The FOV covered approximately 80% of the region between the coronal and lambdoid suture. The first slice of the scan was started approximately 0.318 mm below the coronal suture with a 65 micron increment between slices. A 3-D scan of approximately forty-eight slices was completed for each sample at high resolution (1024×1024 pixels) and a 250 ms integration time. The pixel resolution of the scanned calvaria was approximately 13 microns in all three dimensions. The thresholded image was then skeletonized and the Euclidean Distance Map (EDM) was calculated to determine thickness. This image processing function transforned the binary image into a grey level image where the brightness of each voxel represented the distance to the nearest edge. An estimate of the thickness was calculated by finding the maximum EDM value in each slice (assumed to be the center of the wire) and taking the average and multiplying by two. An adjustment for the curvature of the calvaria was used to determine the actual thickness using the formula, Th_{ACT}=Sin(Theta)* Th_{MEAS}.

The data were analyzed using JMP statistical software (SAS Institute). Treatment effects were initially identified using Student's t-test. P values in FIG. 11 compare all differences between vehicle and treatment. From the data is can be seen that administration of FGF9 caused a significant increase in bone thickness during the experimental period. Indeed this increase in bone was significantly greater than that seen with PGE₂ (a known bone anabolic agent).

### Example 9

Drug Screening Assays

Candidate agents and compounds will be screened for their ability to modulate the expression levels and/or activities of the FGFR3 gene identified as being involved in the differentiation of precursor stem cells into osteoblasts by any technique known to those skilled in the art including those assays described above. In some preferred embodiments, the assay of gene expression level may be conducted using real time PCR. Real time PCR detection may be accomplished by the use of the ABI Prism 7700 Sequence Detection System. The 7700 measures the fluorescence intensity of the sample each cycle and is able to detect the presence of specific amplicons within the PCR reaction. Each sample is assayed for the level of FGFR3 gene expression identified as being involved in the differentiation of precursor cells into osteoblasts.

The expression level of a control gene, for example GAPDH, may be used to normalize the expression levels. Suitable primers for the candidate genes may be selected using techniques well known to those skilled in the art. These primers may be used in conjunction with SYBR green (Molecular Probes), a nonspecific double stranded DNA dye, to measure the expression level MnRNA corresponding to the FGFR3 gene, which will typically be normalized to the GAPDH level in each sample.

Normalized expression levels from cells exposed to the agent are then compared to the normalized expression levels in control cells. Agents that modulate the expression of the FGFR3 gene may be further tested as drug candidates in appropriate in vitro and in vivo models.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

We claim:

1. A method of stimulating a population of stem cells to differentiate into osteoblast cells comprising contacting the population of stem cells with an effective amount of an agent which increases Fibroblast Growth Factor Receptor 3 (FGFR3) expression or activity, wherein the increase in FGFR3 protein expression or activity results in differentiation of the stem cells into osteoblast cells.

2. A method of increasing bone density comprising administering to an animal an effective amount of an agent which increases FGFR3 protein expression, wherein the increase in FGFR3 protein expression increases bone density in the animal.

3. The method of either claim 1 or 2 wherein the stem cell is a mesenchymal stem cell.

4. The method of claim 1 or 2 wherein the agent is selected from the group consisting of an FGF protein, an FGF protein fragment, an FGF-9 protein and an FGF-9 protein fragment.

5. A method of screening for an agent that modulates the differentiation of a population of stem cells into osteoblast cells comprising:
(a) exposing to the stem cells an agent to be tested, and
(b) measuring FGFR3 expression or activity following exposure to the agent,
wherein an increase in FGFR3 expression or activity is indicative of an agent capable of stimulating stem cells to differentiate into osteoblast cells.

6. A method of screening for an agent that increases bone density comprising:
(a) exposing a population of stem cells to the agent; and
(b) measuring FGFR3 expression or activity following exposure to the agent,
wherein an alteration in the level of FGFR3 expression or activity is indicative of an agent capable increasing bone density.

7. A method of screening for an agent capable of ameliorating the effects of osteoporosis comprising:
(a) exposing a population of stem cells expressing FGFR3 to the agent; and
(b) measuring FGFR3 expression or activity following exposure to the agent,
wherein an increase in the level of FGFR3 expression or activity is indicative of an agent capable of ameliorating the effects of osteoporosis.

8. The method of any one of claims 5, 6 or 7 wherein the stem cell is a mesenchymal stein cell.

9. A method of diagnosing a condition characterized by abnormal stem cell differentiation comprising detecting in a stem cell sample the level of FGFR3 expression or activity wherein abnormal FGFR3 expression or activity is indicative of a condition characterized by abnormal stem cell differentiation.

10. A method of diagnosing a condition characterized by abnormal bone density comprising detecting in a stem cell sample the level of FGFR3 expression wherein decreased FGFR3 expression or activity is indicative of a condition characterized by abnormal bone density.

11. A method of diagnosing a condition characterized by an abnormal rate of osteoblast formation comprising detecting in a stem cell sample the level of FGFR3 expression or activity, wherein differential FGFR3 expression or activity is indicative of an abnormal rate of formation of osteoblasts.

12. The method of claim 8, 9 or 10 wherein the condition is osteoporosis.

13. The method of any one of claims 9, 10 or 11 wherein the stem cell is a mesenchymal stem cell.

14. A method of treating a patient with a condition characterized by an abnormal rate of osteoblast formation comprising administering to the patient with decreased FGFR3 expression or activity a pharmaceutical composition which increases FGFR3 expression or activity.

15. A method of treating a patient with a condition characterized by abnormal bone density comprising administering to the patient a pharmaceutical composition which increases FGFR3 expression or activity.

16. A method of treating a patient with osteoporosis comprising administering to the patient a pharmaceutical composition wherein the pharmaceutical composition alters FGFR3 expression or activity.

17. The method of any one of claims 14, 15 or 16 further comprising the step of identifying a patient with decreased FGFR3 expression in a stem cell sample prior to administering the pharmaceutical composition.

18. The method of any one of claims 14, 15 or 16 further comprising the step of comparing FGFR3 expression in the stem cell sample to FGFR3 expression in a stem cell sample from the patient taken before treatment with the pharmaceutical composition.


SEQUENCE LISTING (14 sequences)

SEQ ID NO: 1 - DNA, 3582 bp, Homo sapiens
  CDS (99)..(1679): FGFR3, GenBank Accession No. XM_017699
cagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggccctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctgcccagaggcccaccttcaagcagctggctggaggacctggaccgtgtcttaccgtgacgtccaccgacgagtacctggacctgtcggcgcctttcgagcagtactccccgggtggccaggacacccccagctccagctcctcaggggacgactccgtgtgcccacgacctgctgccccggcccacccagcagtgggggctcgcggacgtgaagggccactggtccccaacaatgtgaggggtcctagcagccaccctgctgctggtgcacagccactcctcggcatgagactcagtgcagatggagagacagctacacagagctttggtctgtgtgtgtgtgtgtgcgtgtgtgtgtgtgtgtgtgcacatccgcgtgtgcctgtgtgcgtgcgcatcttgcctccaggtgcagaggtaccctgggtgtccccgctgctgtgcaacggtctcctgactggtgctgcagcaccgaggggcctttgttctggggggacccagtgcagaatgtaagtgggcccacccggtgggacccccgtggggcagggagctgggcccgacatggctccggcctctgcctttgcaccacgggacatcacagggtgggcctcggcccctcccacacccaaagctgagcctgcagggaagccccacatgtccagcaccttgtgcctggggtgttagtggcaccgcctccccacctccaggctttcccacttcccaccctgcccctcagagactgaaattacgggtacctgaagatgggagcctttaccttttatgcaaaaggtttattccggaaactagtgtacatttctataaatagatgctgtgtatatggtatatatacatatatatatataacatatatggaagaggaaaaggctggtacaacggaggcctgcgaccctgggggcacaggaggcaggcatggccctgggcggggcgtgggggggcgtggagggaggccccagggggtctcacccatgcaagcagaggaccagggccttttctggcaccgcagttttgttttaaaactggacctgtatatttgtaaagctatttatgggcccctggcactcttgttcccacaccccaacacttccagcatttagctggccacatggcggagagttttaatttttaacttattgacaaccgagaaggtttatcccgccgatagagggacggccaagaatgtacgtccagcctgccccggagctggaggatcccctccaagcctaaaaggttgttaatagttggaggtgattccagtgaagatattttatttcctttgtcctttttcaggagaattagatttctataggatttttctttaggagatttattttttggacttcaaagcaagctggtattttcatacaaattcttctaattgctgtgtgtcccaggcagggagacggtttccagggaggggccggccctgtgtgcaggttccgatgttattagatgttacaagtttatatatatctatatatataatttattgagtttttacaagatgtatttgttgtagacttaacacttcttacgcaatgcttctagagttttatagcctggactgctacctttcaaagcttggagggaagccgtgaattcagttggttcgttctgtactgttactgggccctgagtctgggcagctgtcccttgcttgcctgcaggcccatggctcagggtggtctcttcttggggcccagtgcatggtggccagaggtgtcacccaaaccggcaggtgcgattttgttaacccagcgacgaactttccgaaaaataaagacacctggttgctaacctg

SEQ ID NO: 2 - protein, 526 aa, Homo sapiens
MGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMALRPCLTESTLTRVTSGPLGSCSGRSSRWGAPRTPASLWRSSSSC

SEQ ID NO: 3 - DNA, 1417 bp, Homo sapiens
  CDS (178)..(804): FGF9, GenBank Accession No. XM_007105
tgaaacagcagattacttttatttatgcatttaatggattgaagaaaagaaccttttttttctctctctctctgcaactgcagtaagggaggggagttggatatacctcgcctaatatctcctgggttgacaccatcattattgtttattcttgtgctccaaaagccgagtcctctgatggctcccttaggtgaagttgggaactatttcggtgtgcaggatgcggtaccgtttgggaatgtgcccgtgttgccggtggacagcccggttttgttaagtgaccacctgggtcagtccgaagcaggggggctccccaggggacccgcagtcacggacttggatcatttaaaggggattctcaggcggaggcagctatactgcaggactggatttcacttagaaatcttccccaatggtactatccagggaaccaggaaagaccacagccgatttggcattctggaatttatcagtatagcagtgggcctggtcagcattcgaggcgtggacagtggactctacctcgggatgaatgagaagggggagctgtatggatcagaaaaactaacccaagagtgtgtattcagagaacagttcgaagaaaactggtataatacgtactcatcaaacctatataagcacgtggacactggaaggcgatactatgttgcattaaataaagatgggaccccgagagaagggactaggactaaacggcaccagaaattcacacattttttacctagaccagtggaccccgacaaagtacctgaactgtataaggatattctaagccaaagttgacaaagacagtttcttcacttgagcccttaaaaaagtaaccactataaaggtttcacgcggtgggttcttattgattcgctgtgtcatcacatcagctccactgttgccaaactttgtcgcatgcataatgtatgatggaggcttggatgggaatatgctgattttgttctgcacttaaaggcttctcctcctggagggctgcctagggccacttgcttgatttatcatgagagaagaggagagagagagagactgagcgctaggagtgtgtgtatgtgtgtgtgtgtgtgtgtgtgtgtgtgtgtatgtgtgtagcgggagatgtgggcggagcgagagcaaaaggactgcggcctgatgcatgctggaaaaagacacgcttttcatttctgatcagttgtacttcatcctatatcagcacagctgccatacttcgacttatcaggattctggctggtggcctgcgcgagggtgcagtcttacttaaaagactttcagttaattctcactggtatcatcgcagtgaacttaaagcaaagacctcttagtaaaaaataaaaaaaaataaaaaataaaaataaaaaaagttaaatttatttatagaaattccaaa

SEQ ID NO: 4 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 5 - DNA, 55 bp, Artificial Sequence
  Description of Artificial Sequence  Primer for cDNA synthesis
acgtaatacgactcactatagggcgaattgggtcgactttttttttttttttttv

SEQ ID NO: 6 - DNA, 40 bp, Artificial Sequence
  Description of Artificial Sequence  Primer for cDNA synthesis
ctctcaaggatcttaccgctttttttttttttttttttat

SEQ ID NO: 7 - DNA, 40 bp, Artificial Sequence
  Description of Artificial Sequence  Primer for cDNA synthesis
taataccgcgccacatagcattttttttttttttttttcg

SEQ ID NO: 8 - DNA, 40 bp, Artificial Sequence
  Description of Artificial Sequence  Primer for cDNA synthesis
cagggtagacgacgctacgcttttttttttttttttttga

SEQ ID NO: 9 - DNA, 25 bp, Artificial Sequence
  Description of Artificial Sequence  Adapter sequence for cloning
tagcgtccggcgcagcgacggccag

SEQ ID NO: 10 - DNA, 29 bp, Artificial Sequence
  Description of Artificial Sequence  Adapter sequence for cloning
gatcctggccgtcggctgtctgtcggcgc

SEQ ID NO: 11 - DNA, 40 bp, Artificial Sequence
  Description of Artificial Sequence  PCR primer
tgaagccgagacgtcggtcgttttttttttttttttttvn

SEQ ID NO: 12 - DNA, 19 bp, Artificial Sequence
  Description of Artificial Sequence  Forward Q-RT-PCR primer
tggtggccagaggtgtcac

SEQ ID NO: 13 - DNA, 19 bp, Artificial Sequence
  Description of Artificial Sequence  Reverse Q-RT-PCR primer
ggaaagttcgtcgctgggt

SEQ ID NO: 14 - DNA, 23 bp, Artificial Sequence
  Description of Artificial Sequence  TaqMan probe
aaaccggcaggtgcgattttgtt